(19) United States
(12) Patent Application Publication
(10) Pub. No.: US 2025/0389067 A1
(43) Pub. Date: Dec. 25, 2025

(54) BARRIER FABRICS WITH IMPROVED REPELLENCY

(71) Applicant: MAGNERA CORPORATION, Charlotte, NC (US)

(72) Inventors: Lei Wang, Mooresville, NC (US); Nyle Bishop, Mooresville, NC (US); Jonathan T. Nix, Mount Juliet, TN (US)

(21) Appl. No.: 19/243,179

(22) Filed: Jun. 19, 2025

Related U.S. Application Data
(60) Provisional application No. 63/661,643, filed on Jun. 19, 2024.

(51) Int. Cl. D04H 1/413; A61B 46/00; D04H 1/56

(52) U.S. Cl. CPC D04H 1/413; D04H 1/565; A61B 46/40; D10B 2321/022; D10B 2401/02; D10B 2501/04

(57) ABSTRACT

A barrier fabric is provided, in which the barrier fabric includes at least a first nonwoven layer comprising a plurality of first fibers defined by a first polymeric composition. The first polymeric composition includes (i) a first polymer component, and (ii) a first additive component comprising at least one non-fluorinated low-surface tension (NFLST) additives dispersed throughout the first polymeric composition.

### CROSS-REFERENCE TO RELATED APPLICATIONS

This application claims the benefit of U.S. Provisional Application No. 63/661,643, filed on Jun. 19, 2024, which is hereby incorporated by reference in its entirety.

### TECHNICAL FIELD

Embodiments of the presently-disclosed invention relate generally to barrier fabrics comprising at least a first nonwoven layer comprising a plurality of first fibers defined by a first polymeric composition. The first polymeric composition includes (i) a first polymer component, and (ii) a first additive component comprising at least one non-fluorinated low-surface tension (NFLST) additives dispersed throughout the first polymeric composition. The NFLST additive(s) may be incorporated into the plurality of first fibers by use of a masterbatch including the NFLST additive(s). A variety of articles including such barrier fabrics are also provided.

### BACKGROUND

Alcohol repellent fabrics are frequently used in surgical drapes and gowns. These fabrics often consist of barrier fabrics treated with fluoro-chemicals to enhance resistance to penetration by isopropyl alcohol. This has needed in order to meet the industry standards and more precisely to meet the alcohol repellency test. The global regulatory trending desire is to find an approach to eliminate the polyfluorinated (PFC's) treatments. The byproducts generated in the production of the Fluorine Chemical (FC)'s (e.g., perfluorooctanic acid (PFOA) and perfluooctanesulfonic acid (PFOS)), for example, are persistent in the environment and have adverse health effects. In fact, the 2015 Global Suppliers Stewardship phased out of C8 FC and moved to C6 FC to further reduce PFOA and PFOS. Currently, the European Chemicals Agency (ECHA) is in the process of evaluating product lines that use PFC's.

Known Fluoro related chemicals having the lowest critical surface tensions are around 17 mJ/m² (equivalent to dynes/cm). In this regard, a fluoro chemical coated surface has alcohol repellency values of up to 80%˜90% IPA and prevents blood penetration (Synthetic Blood used in ASTM F1670M with Surface Tension: 35-45 dynes/cm). The reduced surface tension imparted by the fluoro chemical is believed to be the mechanism by which the fluoro-chemical coated surfaces exhibit desirable barrier properties to alcohol and blood

Therefore, there at least remains a need in the art for a non-fluorine barrier approaches, such as via an internal or melt additive, to achieve low surface tension surfaces for fibers and their use in the formation of barrier fabrics that may provide similar or improved barrier repellency performance with respect to the penetration of low surface tension fluids (e.g., alcohols and blood).

### SUMMARY OF INVENTION

One or more embodiments of the invention may address one or more of the aforementioned problems. Certain embodiments according to the invention provide a barrier fabric that includes at least a first nonwoven layer comprising a plurality of first fibers defined by a first polymeric composition. The first polymeric composition includes (i) a first polymer component, and (ii) a first additive component comprising at least one non-fluorinated low-surface tension (NFLST) additives dispersed throughout the first polymeric composition.

In another aspect, the present invention provides an article comprising a barrier fabric, such as those described and disclosed herein. The article may comprise, for example, personal protective equipment (PPE) or personal incontinence products (PIP). By way of example only, the article may be a PPE item such as coveralls, leggings, lab coats, aprons, headgear, positive pressure suits, surgical gowns, surgical drapes, pants, jackets, and facemasks. By way of example only, the article may be a PIP item such as feminine hygiene pads, diapers, pull-ups, and adult diapers. The article may also comprise ostomy bags and/or related products, or wound care products (e.g., gauzes or wound dressings).

In another aspect, the present invention provides an article comprising (i) a backsheet comprising a barrier fabric, such as those described and disclosed herein; (ii) a liquid permeable topsheet; and (iii) an absorbent core located between the backsheet and the liquid permeable topsheet. The article may comprise, for example, a surgical gown, a female hygiene article, an underpad, or a diaper.

In another aspect, the present invention provides a method of forming a barrier fabric, such as those described and disclosed herein, in which the method comprises: (i) forming a first polymer melt comprising a first polymeric composition including (a) a first polymer component, and (b) a first additive component, wherein the first additive component includes at least one non-fluorinated low-surface tension (NFLST) additives dispersed throughout the first polymeric composition; (ii) melt spinning the first polymer melt to provide a first plurality of fibers; and (iii) consolidating the first plurality of fibers to provide the barrier fabric.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to the use of lower surface tension chemicals in the polymer melt forming a variety of nonwoven materials, in which the low surface tension chemicals are dispersed throughout the body of the individual fibers forming one or more individual nonwoven layers of the barrier fabric. The lower surface tension chemicals, such as non-fluorinated low-surface tension (NFLST) additives disclosed and described herein, are devoid of fluorine atoms, which may desirably replace the traditional reliance on fluoro-chemicals while achieving similar or improved barrier repellency performance (e.g., prevent the penetration of low surface tension fluids through the fabric). In accordance with certain embodiments of the invention, for example, the fabrics including the NFLST additive(s) exhibit performance comparable to or better than C6 fluorine chemical treated materials with respect to preventing lower surface tension fluid penetration and also passing the blood drop test with synthetic blood in accordance with ASTM F1670M (e.g., 15 minutes of droplet exposure without blood penetration). By way of example only, polypropylene may have a surface tension of 30.5 mJ/m² and polyethylene may have a surface tension of 31.6 mJ/m². By incorporating the NFLST additive(s) within the body of at least some of the fibers forming the barrier fabric, a reduction of the surface tension of the barrier fabric may be achieved due to the NFLST additive(s), which may render the barrier fabric resistant to low surface tension fluid penetration. For example, the inclusion the NFLST additives may reduce the surface tension of polypropylene and/or polyethylene by at least about 10%, such as at least about any of the following: 10, 15, 20, 25, 30, and 35%, and/or at most about any of the following: 75, 70, 65, 60, 55, 50, 45, 40, and 35%.

In accordance with certain embodiments of the invention, the barrier fabric may be made by any method known, such as those described and disclosed herein. Moreover, the barrier fabric may be made from a broad choice of polymeric materials, such as polyolefins (e.g., polypropylene, polyethylene, copolymers thereof, etc.), polyesters, polyamides, natural fibers (e.g., cotton, etc.), and cellulosic fibers (e.g., rayon, wood fibers, etc.). In accordance with certain embodiments of the invention, for example, the barrier fabric comprises a nonwoven material comprising a polyolefin thermoplastic polymer. For example, the barrier fabric may comprise a polypropylene (e.g., polypropylene being defined broadly and includes copolymers and blends containing a polypropylene). In accordance with certain embodiments of the invention, the barrier fabric may comprise a polyethylene (e.g., polyethylene monocomponent fibers, bi-component fibers including a polyethylene component, flash spun polyethylene fibers, etc.).

In accordance with certain embodiments of the invention, the barrier fabric may comprise continuous fibers (e.g., spunbond fibers), staple fibers, fine fibers (e.g., defined broadly to include melt-blown, melt-film fibrillated, electrospun, etc.). As noted above, certain embodiments of the invention may comprise a barrier fabric comprising a layer of cellulosic fibers (e.g., wood pulp) and a layer of synthetic fibers (e.g., thermoplastic polymer) mechanically entangled together (e.g., hydroentangled together). In accordance with certain embodiments of the invention, the barrier fabric may comprise continuous fibers (e.g., spunbond fibers) and fine fibers (e.g., meltblown fibers), such as fabrics having a spunbond-meltblown-spunbond (SMS), such as a SMS or SSMMS structure, where one or several layers of meltblown fibers are sandwiched in between layers of continuous fibers.

The terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The terms “nonwoven” and “nonwoven web”, as used herein, may comprise a web having a structure of individual fibers, filaments, and/or threads that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process. In certain instances, the “nonwoven web” may comprises a plurality of layers, such as one or more spunbond layers and/or one or more meltblown layers. For instance, a “nonwoven web” may comprises a spunbond-meltblown-spunbond structure.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples. Furthermore, the term “consolidated” and “consolidation” may comprise the bonding by means of a through-air-bonding operation. The term “through-air bonded” and “though-air-bonding”, as used herein, may comprise a nonwoven web consolidated by a bonding process in which hot air is used to fuse the fibers at the surface of the web and optionally internally within the web. By way of example only, hot air can either be blown through the web in a conveyorized oven or sucked through the web as it passes over a porous drum as a vacuum is developed. The temperature of and the rate of hot air are parameters that may determine the level or the extent of bonding in nonwoven web. In accordance with certain embodiments of the invention, the temperature of the hot air may be high enough to melt, induce flowing, and/or fuse the a plurality of fibers having a lower melting point temperature or onset of lower melting point temperature (e.g., amorphous fibers) to a plurality of fibers having a higher melting point temperature or onset of lower melting point temperature (e.g., semi-crystalline or crystalline fibers). Such a web may be considered a “consolidated nonwoven”, “nonwoven fabric” or simply as a “fabric” according to certain embodiments of the invention.

The term “layer”, as used herein, may comprise a generally recognizable combination of similar material types and/or functions existing in the X-Y plane.

The term “spunbond”, as used herein, may comprise fibers which are formed by extruding molten thermoplastic material as filaments from a plurality of fine, usually circular, capillaries of a spinneret with the diameter of the extruded filaments then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

The term “melt fibrillation”, as used herein, may comprise a general class of making fibers defined in that one or more polymers are molten and may be extruded into many possible configurations (e.g. co-extrusion, homogeneous or bicomponent films or filaments) and then fibrillated or fiberized into a plurality of individual filaments for the formation of melt-fibrillated fibers. Non limiting examples of melt-fibrillation methods may include melt blowing, melt fiber bursting, and melt film fibrillation. The term “melt-film fibrillation”, as used herein, may comprise a method in which a melt film is produced from a melt and then a fluid is used to form fibers (e.g., melt-film fibrillated fibers) from the melt film. Examples include U.S. Pat. Nos. 6,315,806, 5,183,670, 4,536,361, 6,382,526, 6,520,425, and 6,695,992, in which the contents of each are incorporated by reference herein to the extent that such disclosures are consistent with the present disclosure. Additional examples include U.S. Pat. Nos. 7,628,941, 7,722,347, 7,666,343, 7,931,457, 8,512,626, and 8,962,501, which describe the Arium™ melt-film fibrillation process for producing melt-film fibrillated fibers (e.g., having sub-micron fibers).

The term “fluorochemical”, as used herein, may comprise any of various chemical compounds containing fluorine, particularly organic compounds (e.g., fluorocarbons such as perfluoroalkanes) in which fluorine has replaced a large proportion of the hydrogen attached to the carbons. Fluorochemicals may exhibit low surface tension and low viscosity and are extremely stable due to the strength of the carbon-fluorine bond. Fluorochemicals are not miscible with most organic solvents.

The term “dry basis”, as used herein may comprise the calculation or measurement of a weight percentage in which the presence of water and/or other solvents (e.g., alcohols) are ignored or excluded for purposes of the calculation or measurement. Weight percentages may frequently be measured on a dry basis to remove the effects of evaporation and/or condensation which may happen naturally throughout the useful life of a composition or article.

The term “cellulosic fiber”, as used herein, may comprise fibers derived from hardwood trees, softwood trees, or a combination of hardwood and softwood trees prepared for use in, for example, a papermaking furnish and/or fluff pulp furnish by any known suitable digestion, refining, and bleaching operations. The cellulosic fibers may comprise recycled fibers and/or virgin fibers. Recycled fibers differ from virgin fibers in that the fibers have gone through the drying process at least once. In certain embodiments, at least a portion of the cellulosic fibers may be provided from non-woody herbaceous plants including, but not limited to, kenaf, cotton, hemp, jute, flax, sisal, or abaca. Cellulosic fibers may, in certain embodiments of the invention, comprise either bleached or unbleached pulp fiber such as high yield pulps and/or mechanical pulps such as thermo-mechanical pulping (TMP), chemical-mechanical pulp (CMP), and bleached chemical-thermo-mechanical pulp BCTMP. In this regard, the term “pulp”, as used herein, may comprise cellulose that has been subjected to processing treatments, such as thermal, chemical, and/or mechanical treatments. Cellulosic fibers, according to certain embodiments of the invention, may comprise one or more pulp materials.

All whole number end points disclosed herein that can create a smaller range within a given range disclosed herein are within the scope of certain embodiments of the invention. By way of example, a disclosure of from about 10 to about 15 includes the disclosure of intermediate ranges, for example, of: from about 10 to about 11; from about 10 to about 12; from about 13 to about 15; from about 14 to about 15; etc. Moreover, all single decimal (e.g., numbers reported to the nearest tenth) end points that can create a smaller range within a given range disclosed herein are within the scope of certain embodiments of the invention. By way of example, a disclosure of from about 1.5 to about 2.0 includes the disclosure of intermediate ranges, for example, of: from about 1.5 to about 1.6; from about 1.5 to about 1.7; from about 1.7 to about 1.8; etc.

Certain embodiments according to the invention provide barrier fabric comprising a barrier fabric that includes at least a first nonwoven layer comprising a plurality of first fibers defined by a first polymeric composition. The first polymeric composition includes (i) a first polymer component, and (ii) a first additive component comprising at least one non-fluorinated low-surface tension (NFLST) additives dispersed throughout the first polymeric composition. In this regard the NFLST additive(s) may include a single such additive or a combination of several different types of such additives, such as those described and disclosed herein. In accordance with certain embodiments of the invention a “polymer component” (e.g., a first polymer component, second polymer component, third polymer components, etc.) as used herein by comprise a polymer or polymer blend that forms a matrix component of the fiber(s) and the “additive component” as used herein may be other components, which may be devoid of polymers or include polymers different than those used to form the matrix component, dispersed throughout the matrix component/polymer component.

In accordance with certain embodiments of the invention, the NFLST additive(s) may include one or more waxes, such as a paraffin wax, a glycerol tristearate, a beeswax, a cuticular wax, or any combination thereof. Persons having ordinary skill in the art understand that many waxes, particularly naturally occurring waxes, include a combination of individual components. For example, naturally occurring beeswax includes palmitate, palmitoleate, and oleate esters of long-chain (e.g. 30-32 carbons) aliphatic alcohols, with each individual component being a “component thereof” in relation to beeswax. For ease of reference, the term “wax or component thereof” may be collectively referred to as “wax” throughout the remaining description. In accordance with certain embodiments of the invention, the one or more waxes may include from about 30 to about 80 carbon atoms, such as at least about any of the following: 30, 32, 35, 38, 40, 42, 45, 48, and 50 carbon atoms, and/or at most about any of the following: 80, 75, 70, 65, 60, 58, 57, 56, 55, 52, and 50 carbon atoms. Additionally or alternatively, the one or more waxes may have an acid value of from 0.1 mg to 220 mg, KOH/g as measured in accordance with the Enterprise Standard Test, such as at least about any of the following: 0.1, 0.2, 0.3, 0.5, 0.8, 1, 1.5, 2, 2.5, 3, 5, 8, 10, 15, 20, 30, 40, 50, 60, 80, and 100 mg, KOH/g as measured in accordance with the Enterprise Standard Test, and/or at most about any of the following: 220, 200, 180, 160, 150, 140, 120, and 100 mg, KOH/g as measured in accordance with the Enterprise Standard Test. In accordance with certain embodiments of the invention, the one or more waxes may have an acid value of from 0.1 mg to 5 mg, KOH/g as measured in accordance with the Enterprise Standard Test, such as at least about any of the following: 0.1, 0.2, 0.3, 0.4, 0.5, 0.6, 0.8, and 1 mg, KOH/g as measured in accordance with the Enterprise Standard Test, and/or at most about any of the following: 5, 4.5, 4, 3.5, 3, 2.5, 2, 1.5, and 1 mg, KOH/g as measured in accordance with the Enterprise Standard Test.

Cuticular wax, for example, plays a major role in the growth and storage of plant fruits. The cuticular wax coating, which covers the outermost layer of a fruit's epidermal cells, is insoluble in water. Cuticular wax is mainly composed of very long-chain fatty acids (VLCFAs); their derivatives, including esters, primary alcohols, secondary alcohols, aldehydes, and ketones; and triterpenoids. This complex mixture of lipids is probably biosynthesized in the epidermal cells of most plants and exuded onto the surface. Cuticular wax not only makes the fruit less susceptible to microbial infection but also reduces mechanical damage to the fruit, thereby maintaining the fruit's commodity value. To date, research has mostly focused on the changes, function, and regulation of fruit wax before harvest, while ignoring the changes and functions of wax in fruit storage.

Beeswax includes hydrocarbons (12%-16%) with a predominant chain length of C27-C33, mainly heptacosane, nonacosane, hentriacontane, pentacosane and tricosane; free fatty acids (12%-14%), with a chain length of C24-C32; free fatty alcohols (ca. 1%) of C28-C35; linear wax monoesters and hydroxymonoesters (35%-45%) with chain lengths generally of C40-C48, derived fundamentally from palmitic, 15-hydroxypalmitic and oleic acids; complex wax esters (15%-27%) containing 15-hydroxypalmitic acid or diols, which through their hydroxyl group, are linked to another fatty-acid molecule; exogenous substances that are mainly residues of propolis, pollen, small pieces of floral component factors and pollution.

In accordance with certain embodiments of the invention, the NFLST additive(s) may include (additionally or alternatively) an organo-modified siloxane, such as a polydimethylsiloxane (PDMS), having one or more organic groups grafted onto one or both chain ends or onto the backbone, wherein the one or more organic groups may include acrylates, such as a methacrylate group, a methyl methacrylate group, an ethyl acrylate group, a cyanoacrylate group, a poly(methyl acrylate) group, a poly(ethyl acrylate) group, a poly(butyl acrylate) group, or combinations thereof.

In accordance with certain embodiments of the invention, the organo-modified siloxane is selected from Formula (I):

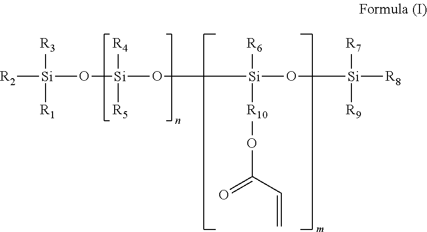

wherein,
R1-R9 are independently selected from —H, a C1-C10 radical, —OH, alkoxy radical, and an acrylate functional group,
R10 is selected from a C1-C10 hydrocarbon;
n is selected from 1 to about 100, such as at least about any of the following: 1, 3, 5, 10, 20, 30, 40, and 50, and/or at most about any of the following: 100, 90, 80, 70, 60, and 50, and
m is selected from 1 to about 100, such as at least about any of the following: 1, 3, 5, 10, 20, 30, 40, and 50, and/or at most about any of the following: 100, 90, 80, 70, 60, and 50.

In accordance with certain embodiments of the invention the acrylate group, for example, from Formula (I) may be a methacrylate group. Additionally or alternatively, one or more of R1-R10 may include an acrylate or methacrylate group.

In accordance with certain embodiments of the invention, the organo-modified siloxane is selected from Formula (II):

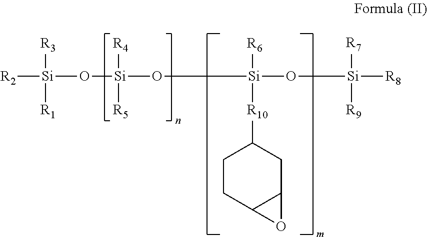

wherein,
R1-R9 are independently selected from —H, a C1-C10 radical, —OH, alkoxy radical, and an acrylate functional group,
R10 is selected from a C1-C10 hydrocarbon;
n is selected from 1 to about 100, such as at least about any of the following: 1, 3, 5, 10, 20, 30, 40, and 50, and/or at most about any of the following: 100, 90, 80, 70, 60, and 50, and
m is selected from 1 to about 100, such as at least about any of the following: 1, 3, 5, 10, 20, 30, 40, and 50, and/or at most about any of the following: 100, 90, 80, 70, 60, and 50.

In accordance with certain embodiments of the invention, the NFLST additive(s) may include (additionally or alternatively) an acrylic-functional polymer including an alkyl silane methacrylate group. By way of example, the acrylic-functional polymer may be anionic, cationic, or non-ionic. In accordance with certain embodiments of the invention, the alkyl portion(s) of the acrylic-functional polymer may independently comprise from 1 to about 20 carbon atoms, such as at least about any of the following: 1, 2, 3, 4, 5, 6, 7, 8, 9, and 10 carbon atoms, and/or at most about any of the following: 20, 19, 18, 17, 16, 15, 14, 13, 12, 11, and 10 carbon atoms

In accordance with certain embodiments of the invention, the NFLST additive(s) may include (additionally or alternatively) a composition including (a) a wax or a component thereof having an acid value of from 0.1 mg to 220 mg, KOH/g as measured in accordance with the Enterprise Standard Test, such as at least about any of the following: 0.1, 0.2, 0.3, 0.5, 0.8, 1, 1.5, 2, 2.5, 3, 5, 8, 10, 15, 20, 30, 40, 50, 60, 80, and 100 mg, KOH/g as measured in accordance with the Enterprise Standard Test and/or at most about any of the following: 220, 200, 180, 160, 150, 140, 120, and 100 mg, KOH/g as measured in accordance with the Enterprise Standard Test and (b) a retention aid comprising a nitrogen-containing polymer independently selected from the group consisting of:
(i) a nitrogen-containing polymer of Formula (III),

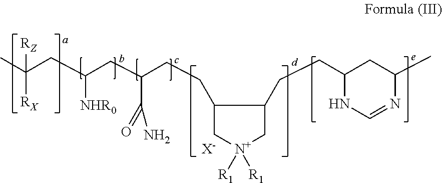

wherein ‘a’, ‘b, ‘c’, ‘d’, and ‘e’ individually represent the molar percent of each repeating unit included in the nitrogen-containing polymer of Formula (III),
wherein R₀ is independently selected from the group consisting of:

### of H,

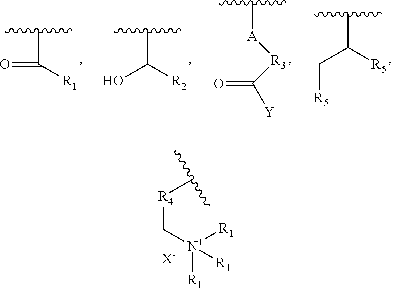

and combinations thereof, and
wherein:
   R_{z} is independently selected from H, —CH₃, and combinations thereof,
   Rₓ is independently selected from H, —OH, —COOH, —COOR₁, —OCOR₁, —R₁, —R₃OH, —OR₁, —NR₁R₁, —R₃NH₂, —NH₂, —COO(CH₂)₂N(R₁)₂, —COO(CH₂)₂N+(R₁)₃X⁻, —COO(CH₂)₃N+(R₁)₃X⁻, and combinations thereof, with the proviso that when Rₓ is —NH₂, R_{z} is —CH₃,
   Y is independently selected from H, —OH, —R₁, —OR₁, —NR₁R₁, —NH₂, and combinations thereof,
   Z is independently selected from H, —OH, —C═O, —R₁, —OR₁, —NR₁R₁, —NH₂ and combinations thereof,
   R₁ is independently selected from H, a straight chain or branched alkyl or alkenyl containing up to 22 carbons, and combinations thereof,
   R₂ is independently selected from H, a monosaccharide, an oligosaccharide, polysaccharide moiety, a straight or branched alkyl or alkenyl group up to 22 carbons optionally containing a hydroxyl or aldehyde group, and combinations thereof,
   R₃ is independently selected from a straight chain or branched alkyl or alkenyl containing up to 22 carbons or combinations thereof,
   R₄ is independently selected from a straight chain or branched alkyl group containing up to 18 carbons, optionally substituted with a hydroxyl group, and combinations thereof,
   R₅ is independently selected from H, —OH, —COOH, —COOR₁, —OCOR₁, —R₁, —R₁OH, —OR₁, —CONH₂, —CONHCHOHCHO, —NR₁, —NR₁R₁, —R₁NH₂, —NH₂, and combinations thereof,
   A is independently selected from C═O, —CH₂, and combinations thereof, and
   X⁻ is independently an anion;
ii. a polyethyleneimine;
iii. a polyaminoamide;
iv. a copolymer formed from the reaction product of epichlorohydrin and dimethylamine; and
v. combinations thereof.

In accordance with certain embodiments of the invention, ‘a’, ‘b, ‘c’, ‘d’, and ‘e’ of Formula (III) may each independently from each other have a value from 0 to 100 mol. %, such as at least about any of the following: 0, 1, 3, 5, 8, 10, 15, 20, 25, 30, 35, 40, 45, and 50 mol. %, and/or at most about any of the following: 100, 95, 90, 85, 80, 75, 70, 65, 60, 55, and 50 mol. %.

Persons having ordinary skill in the art understand that many waxes, particularly naturally occurring waxes, include a combination of individual components. For example, naturally occurring beeswax includes palmitate, palmitoleate, and oleate esters of long-chain (e.g. 30-32 carbons) aliphatic alcohols, with each individual component being a “component thereof” in relation to beeswax. For ease of reference, the term “wax or component thereof” may be collectively referred to as “wax” throughout the remaining description.

Although the wax may not be limited to any particular wax in accordance with certain embodiments of the invention, typically the wax may be selected from a group consisting of a stearate, beeswax (both synthetic and natural), candelilla wax, palmitate, behenate, and combinations thereof. For example, the wax of the sizing agent may be beeswax or a stearate, or both. Alternatively, the wax may be behenate or palmitate, or both.

In accordance with certain embodiments of the invention, the NFLST additive(s) may comprise from about 0.5 to about 30% by weight of the first polymeric composition, such as at least about any of the following: 0.5, 0.8, 1, 1.5, 2, 5, 8, 10, 12, and 15% by weight of the first polymeric composition, and/or at most about any of the following: 30, 28, 25, 22, 20, 18, and 15% by weight of the first polymeric composition. Additionally or alternatively, the first plurality of fibers comprise spunbond fibers, meltblown fibers, or staple fibers.

The barrier fabric, in accordance with certain embodiments of the invention, include the first nonwoven layer that is a first spunbond layer defining a first outermost layer, and wherein the first plurality of fibers comprise a first plurality of continuous spunbond fibers. The barrier fabric may further comprise (i) a second nonwoven layer comprising a second spunbond layer defining a second outermost layer including a second plurality of continuous spunbond fibers, and (ii) a plurality of inner fine fiber-containing nonwoven layers including a first fine fiber-containing layer including a first plurality of fine fibers, wherein the plurality of inner fine fiber-containing nonwoven layers are located directly or indirectly between the first spunbond layer and the second spunbond layer. By way of example, the second spunbond layer may include a second polymeric composition including (i) a second polymer component, and (ii) a second additive component comprising at least one NFLST additive dispersed throughout the second polymeric composition. Additionally or alternatively, the first fine fiber-containing layer may include a third polymeric composition including (i) a third polymer component, and (ii) a third additive component comprising at least one NFLST additive dispersed throughout the third polymeric composition. The first plurality of fine fibers may comprises meltblown fibers, melt-fibrillated fibers, or electrospun fibers.

In this regard, any combination or variation of the individual nonwoven layers of the barrier fabric may include the NFLST additve(s) dispersed throughout the body of the respective fibers for a respective individual nonwoven layer. By way of example only, each individual layer of the barrier fabric includes a respective amount of at least one NFLST additive dispersed throughout the respective polymeric composition of the fibers of each respective individual layer.

In accordance with certain embodiments of the invention, the barrier fabric may comprise one of the following structures:
(Structure 1) S1ₐ-M_{b}-S2_{c};
(Structure 2) S1ₐ-N_{d}—S2_{c};
(Structure 3) S1ₐ-M_{b}-N_{d}—S2_{c};
(Structure 4) S1ₐ-N_{d}-M_{b}-N_{d}—S2_{c}
(Structure 5) S1ₐ-M_{b}-N_{d}-M_{b}-S2_{c}; or any combinations thereof;
Wherein
‘M’ comprises a meltblown layer or a melt-fibrillated layer;
‘N’ comprises a sub-micron fiber-containing layer, such as electrospun fibers;
‘S1’ comprises a first spunbond layer;
‘S2’ comprises a second spunbond layer;
‘a’ represents the number of layers and is independently selected from 1, 2, 3, 4, and 5;
‘b’ represents the number of layers is independently selected from 1, 2, 3, 4, 5, 6, 7, and 8;
‘c’ represents the number of layers is independently selected from 1, 2, 3, 4, and 5; and
‘d’ represents the number of layers is independently selected from 1, 2, 3, 4, 5, 6, 7, and 8;
‘e’ represents the number of layers is independently selected from 1, 2, 3, 4, and 5.

The barrier fabric, in accordance with certain embodiments of the invention, may have a structure according to Structure 1, and wherein ‘a’ is 1 or 2, ‘b’ is 3, 4, or 5, and ‘c’ is 1 or 2. As noted above, at least one of the individual nonwoven layers includes the NFLST additve(s) dispersed throughout the body of the respective fibers for a respective individual nonwoven layer. By way of example, only the meltblown layer may include the NFLST additive(s). Alternatively, only the spunbond layers may include the NFLST additive(s). Alternatively, each and every layer may include the NFLST additive(s).

In accordance with certain embodiments of the invention, the NFLST additive(s) may comprises a first ratio between a total wax content and a total organo-modified siloxane content on a dry basis (% wt.:% wt.) from about 1:10 to about 10:1, such as at least about any of the following: 1:10, 1:8, 1:5, 1:2, and 1:1, and/or at most about any of the following: 10:1, 8:1, 5:1, 2:1 and 1:1.

In accordance with certain embodiments of the invention, the NFLST additive(s) may comprise a second ratio between a total wax content and a total acrylic-functional polymer including an alkyl silane methacrylate group content on a dry basis (% wt.:% wt.) from about 1:10 to about 10:1, such as at least about any of the following: 1:10, 1:8, 1:5, 1:2, and 1:1, and/or at most about any of the following: 10:1, 8:1, 5:1, 2:1 and 1:1.

In accordance with certain embodiments of the invention, the NFLST additive(s) may comprise a third ratio between a total organo-modified siloxane content and a total acrylic-functional polymer including an alkyl silane methacrylate group content on a dry basis (% wt.:% wt.) from about 1:10 to about 10:1, such as at least about any of the following: 1:10, 1:8, 1:5, 1:2, and 1:1, and/or at most about any of the following: 10:1, 8:1, 5:1, 2:1 and 1:1.

In accordance with certain embodiments of the invention, the first additive component (or any additive component of any respective nonwoven layer) may optionally comprise a wetting agent (e.g., one or more surfactants), such as a cationic wetting agent, an anionic wetting agent, a non-ionic wetting agent, or any combination thereof. The wetting agent may comprise from about 0.1 to about 5% by weight of the first polymeric composition (e.g., or respective polymeric composition) on a dry basis, such as at least about any of the following: 0.1, 0.5, 0.8, 1, 1.5, 2, 2.5, and 3% by weight of the first polymeric composition (e.g., or respective polymeric composition) on a dry basis, and/or at most about any of the following: 5, 4, and 3% by weight of the first polymeric composition (e.g., or respective polymeric composition) on a dry basis.

In accordance with certain embodiments of the invention, the barrier fabric may optionally include a topical coating comprising a non-fluorinated barrier coating (NFBC), wherein the NFBC comprises one or more NFLST additives as described and disclosed herein. The NFBC, for example, may include an alkyl urethane emulsion, and/or a paraffin wax (or other waxes disclosed herein) emulsion and/or an acrylic (acrylate) copolymer emulsion. The topical coating may be applied to enhance the barrier/repellency properties of the barrier fabric.

In accordance with certain embodiments of the invention, the barrier fabric may comprise an antistatic composition located on a first outermost surface, a second outermost surface, or both. In accordance with certain embodiments of the invention, the antistatic composition may be located on at least a portion of the first outermost surface and also on at least the second outermost surface. The first outermost surface may have the antistatic composition on one or more separate and discrete locations or, alternatively, be completely coated with the antistatic composition. In accordance with certain embodiments of the invention, the second outermost surface may have the antistatic composition on one or more separate and discrete locations or, alternatively, be completely coated with the antistatic composition. In accordance with certain embodiments of the invention, the antistatic composition comprises at least one antistatic agent, in which the antistatic composition comprises at least one of a non-ionic antistatic agent, an anionic antistatic agent, a cationic antistatic agent, an amphoteric antistatic agent, or any combination thereof. In accordance with certain embodiments of the invention, the at least one antistatic agent comprises an alkylphosphate or a phosphate ester.

In accordance with certain embodiments of the invention, the barrier fabric may comprise at least one binder. The at least one binder (if present), for instance, may comprise an anionic binder, a cationic binder, non-ionic binder, an amphoteric binder, or any combinations thereof. The binder, in accordance with certain embodiments of the invention, may comprise binder agents that are self-cross linking chemicals (e.g., self-crosslinking non-ionic binder) that improve barrier properties. In accordance with certain embodiments of the invention, the binder may comprise an ethylene vinyl acetate copolymer emulsion, an acrylic emulsion, a vinyl acrylic emulsion, or combinations thereof. For example, the at least one binder may comprise at least one of an acrylic binder, a styrene-butadiene rubber binder, a vinyl copolymer binder, a vinyl acetate binder, an ethylene vinyl acetate binder, a polyvinyl chloride binder, a polyurethane binder, or any combination thereof. In accordance with certain embodiments of the invention, the at least one binder comprises an acrylic binder, such as an anionic acrylic binder, a cationic acrylic binder, or a non-ionic acrylic binder. The binder agent(s), in accordance with certain embodiments of the invention, may comprise from about 0.1 to about 10% by weight of the basis weight of the barrier fabric on a dry basis, such as at least about any of the following: 0.1, 0.5, 0.8, 1, 1.5, 2, 2.5, and 3% by weight of the basis weight of the barrier fabric on a dry basis, and/or at most about any of the following: 10, 9, 8, 7, 6, 5, 4, and 3% by weight of the basis weight of the barrier fabric on a dry basis. For example only, if the barrier fabric has a basis weight of 100 gsm, 5% by weight of the basis weight of the barrier fabric is 5 gsm. In accordance with certain embodiments of the invention, however, the barrier fabric may be devoid of a binder.

In accordance with certain embodiments of the invention, the barrier fabric is devoid of any fluorine atoms. For instance, the barrier fabric may be devoid of any melt additives or topical additives that contains fluorine atoms.

The barrier fabric, in accordance with certain embodiments of the invention, may have a hydrohead from about 40 mbar, such as at least about any of the following: 40, 50, 60, 70, 80, 90, 100, 110, 120, 130, 140, and 150 mbar and/or at most about any of the following: 250, 220, 200, 180, 160, and 150 mbar. Additionally or alternatively, the barrier fabric may have an air permeability of 5 CFM or greater according to IST70.1, such as at least about any of the following: 5, 10, 30, 50, 80, 100, 150, and 200 CFM, and/or at most about any of the following: 500, 450, 400, 350, 300, 250, and 200 according to IST70.1. Additionally or alternatively, the barrier fabric may have a liquid strike through time (LSTST) from about 5 to about 1500 seconds, such as at least about any of the following: 5, 10, 20, 30, 50, 60, 80, 100, 120, 150, 200, 250, 300, 350, 400, 450, 500, 550, and 600 seconds, and/or at most about any of the following: 1500, 1400, 1200, 1100, 1000, 980, 950, 920, 900, 880, 850, 820, 800, 780, 750, 720, 700, 680, 650, 620, 600 seconds. In accordance with certain embodiments of the invention, the barrier fabric may have a LSTST of 1000 seconds or larger. Additionally or alternatively, the barrier fabric may have an IPA repellency % from about 20 to about 60%, such as at least about any of the following: 20, 30, and 40% and/or at most about any of the following: 60, 50, and 40%. Additionally or alternatively, the barrier fabric may have an ethanol repellency % from about 40 to about 90%, such as at least about any of the following: 40, 50, and 60% and/or at most about any of the following: 90, 80, 70, and 60%.

The barrier fabric, in accordance with certain embodiments of the invention, may have a liquid absorptive capacity from about 10 to about 30% as determined in accordance with NWSP 10.1.R0(20) (Clause b) following Section 8.2: The liquid absorptive capacity and using a 0.9% NaCl solution, such as at least about any of the following 10, 12, 15, and 18%, and/or at most about any of the following: 30, 28, 25, 22, 20, and 18%. Additionally or alternatively, the barrier fabric may have a liquid absorptive capacity from about 100 to about 300% as determined in accordance with NWSP 10.1.R0(20) (Clause b) following Section 8.2: The liquid absorptive capacity and using a Triton-X-100 (32 dyne surface tension) solution, such as at least about any of the following 100, 120, 150, 180, and 200%, and/or at most about any of the following: 300, 280, 250, 220, and 200%.

The barrier fabric, in accordance with certain embodiments of the invention, may include a plurality of discrete bond sites defining a bonded area. The discrete (e.g., individual or separate) bond sites may be thermal bond sites and/or ultrasonic bond sites. In this regard, the bonded area, for example, may comprise from about 8 to about 40%, such as at least about any of the following: 8, 10, 12, 15, 18, 20, 22, and 25%, and/or at most about any of the following: 40, 35, 30, 28, and 25%.

In accordance with certain embodiments of the invention, the barrier fabric may have a basis weight from about 5 to about 300 gsm, such as at least about any of the following: 5, 6, 8, 10, 15, 20, 25, 30, 34, 40, 45, 50, 60, 80, and 100 gsm, and/or at most about any of the following: 300, 280, 270, 250, 220, 200, 180, 150, 120, and 100 gsm.

In another aspect, the present invention provides an article comprising a barrier fabric, such as those described and disclosed herein. The article may comprise, for example, personal protective equipment (PPE) or personal incontinence products (PIP). By way of example only, the article may be a PPE item such as coveralls, leggings, lab coats, aprons, headgear, positive pressure suits, surgical gowns, surgical drapes, pants, jackets, and facemasks. By way of example only, the article may be a PIP item such as feminine hygiene pads, diapers, pull-ups, and adult diapers. The article may also comprise ostomy bags and/or related products, or wound care products (e.g., gauzes or wound dressings).

In another aspect, the present invention provides an article comprising (i) a backsheet comprising a barrier fabric, such as those described and disclosed herein; (ii) a liquid permeable topsheet; and (iii) an absorbent core located between the backsheet and the liquid permeable topsheet. The article may comprise, for example, a surgical gown, a female hygiene article, an underpad, or a diaper.

Ostomy bags and/or related products, and wound care products (e.g., gauzes or wound dressings), in accordance with certain embodiments of the invention, the barrier fabric, such as those described and disclosed herein, may exhibit an improved drying rate and/or time as compared to non-treated fabrics having an identical construction (e.g., same polymer system, same basis weight, same fiber types and sizes, etc.) but lacking the NFLST additive(s). In accordance with certain embodiments of the invention, the ability of a nonwoven fabric (e.g., barrier fabric), such as those described and disclosed herein having one or more NFLST additives, to dry after bathing or cleaning a user's skin around the area where, for example, the ostomy bag is in contact with the skin is particularly important. In this regard, ostomy bags may comprise at least an outermost surface at least partially defined by a barrier fabric as described and disclosed herein. For instance, the barrier fabric, such as those described and disclosed herein, forming all or a portion of an ostomy bag adjacent and/or proximate to a user's skin may have a drying time.

For example, ostomy bags, in accordance with certain embodiments of the invention, may include an outermost layer of a nonwoven fabric, such as those described and disclosed herein, and an interior film layer that prevents liquid or fecal matter from passing through the body of the ostomy bag. The nonwoven fabric and the interior film layer (e.g., film layer) may be bonded to each other via a variety of techniques, for example, adhesively bonded, ultrasonically bonded, thermally bonded, or the film layer may be melt extruded directly onto the nonwoven fabric. The film layer may not be particularly limited as long as the film provides adequate liquid and/or gaseous barrier properties to prevent undesirable passage of liquids and/or gases from inside the ostomy bag to an external environment. In accordance with certain embodiments of the invention, the nonwoven fabric may simply comprises a spunbond nonwoven fabric (e.g., including from 1 to 10 individual spunbond layers) including at least one NFLST additive therein. Such nonwoven fabrics may have a smaller barrier effect (e.g., lower hydrostatic head, alcohol repellency, etc.) compared to nonwoven fabrics having a general spunbond-meltblown-spunbond structure. Alternatively, the nonwoven fabric may comprise a general spunbond-meltblown-spunbond structure, such as those described and disclosed herein, that provide a larger barrier effect (e.g., larger hydrostatic head, alcohol repellency, etc.) compared to a simply spunbond nonwoven fabric. In this regard, the relative “barrier effect” of the nonwoven fabric utilized in an ostomy bag (or wound care article) may be varied as desired. For example, nonwoven fabrics having a larger the barrier effect (e.g., SMS-type nonwoven fabrics) may prevent a capillary effect between the nonwoven fabric and the film layer while nonwoven fabrics having a smaller barrier effect (e.g., spunbond nonwoven fabric) may allow fluid to drain from an interface between the nonwoven fabric and the film layer.

In another aspect, the present invention provides a method of forming a barrier fabric, such as those described and disclosed herein, in which the method comprises: (i) forming a first polymer melt comprising a first polymeric composition including (a) a first polymer component, and (b) a first additive component, wherein the first additive component includes at least one non-fluorinated low-surface tension (NFLST) additives dispersed throughout the first polymeric composition; (ii) meltspinning the first polymer melt to provide a first plurality of fibers; and (iii) consolidating the first plurality of fibers to provide the barrier fabric.

In accordance with certain embodiments of the invention, the step of forming a first polymer melt comprises adding a master batch including the at least one NFLST additives to the polymer component. The first plurality of fibers, as noted above, may comprise continuous spunbond fiber or meltblown fibers. In this regard, the step of meltspinning the first polymer melt to provide a first plurality of fibers may be a spunbond process or a meltblown process.

In accordance with certain embodiments of the invention, the step of meltspinning the first polymer melt to provide a first plurality of fibers is a spunbond process, and the method may further comprise (i) forming a second polymer melt comprising a second polymeric composition including (a) a second polymer component, and (b) a second additive component, wherein the second additive component includes at least one non-fluorinated low-surface tension (NFLST) additives dispersed throughout the second polymeric composition, and (ii) meltspinning the second polymer melt via a spunbond process to form a second plurality of continuous spunbond fibers. The method may also comprise (i) forming a third polymer melt comprising a third polymeric composition including (a) a third polymer component, and (b) a third additive component, wherein the third additive component includes at least one non-fluorinated low-surface tension (NFLST) additives dispersed throughout the third polymeric composition, and (ii) meltspinning the second polymer melt via a meltblown process to form a plurality of meltblown fibers. In accordance with certain embodiments of the invention, the method may comprise a step of depositing the plurality of meltblown fibers onto the first plurality of continuous spunbond fibers, and depositing the second plurality of continuous spunbond fibers onto the plurality of meltblown fibers. Additionally, the consolidating step may comprise bonding each of the individual layers present together via a thermal bonding operation and/or an ultrasonic bonding operation. In accordance with certain embodiments of the invention, the thermal bonding operation and/or ultrasonic bonding operation forms a plurality of discrete bond sites defining a bonded area. The bonded area, for example, may comprise from about 8 to about 40%, such as at least about any of the following: 8, 10, 12, 15, 18, 20, 22, and 25%, and/or at most about any of the following: 40, 35, 30, 28, and 25%.

### EXAMPLES

The present disclosure is further illustrated by the following examples, which in no way should be construed as being limiting. That is, the specific features described in the following examples are merely illustrative and not limiting.

### A. Test Methods

Basis weight of the following examples was measured according to ASTM test method D3776. The results were provided in units of mass per unit area in g/m² (gsm).

Alcohol repellency of the following examples was measured according to test method IST 80.8

Hydrohead of the following examples was measured according to standard test method IST 80.8 and ramping up the pressure at a rate of 60 mbar/min. A larger hydrohead value is more desirable for increased the barrier performance.

Air Permeability is a measure of air flow passing through a sheet under at a stated pressure differential between the surfaces of the sheet and was conducted according to ASTM D 737, Test area 38 cm², Test Pressure @ 125 Pa, and is reported in ml/dm²/min. A larger air permeability value is indicative of improved comfort for surgical gown and drape applications.

Low Surface Tension Strikethrough Time (LSTST) is a test that determines the time it takes for a particular quantity of liquid discharged at a prescribed rate to fully penetrate a sample of a nonwoven fabric. The method employed herein is a modification to WSP 70.3 (05). The changes were as follows: the test liquid was a 32 mN/m surface tension liquid prepared with Triton-X-100 and distilled water.

### B. Spunbond Barrier/Repellency

The lower surface tension fluoro-free additive chemical & masterbatch treated nonwoven fabrics will have longer surface tension strikethrough time and alcohol repellency, such as around 50% IPA or 70%˜80% Ethanol repellency or higher as well as prevent blood penetration.

Polypropylene resin carrier was mixed with different NFLST additives and varying loadings, in which the mixing temperature was ˜2200° C. for spunbond lines and for fiber spinning and ˜2600° C. for meltblown lines and for fiber spinning. The melt additive (NFLST additives) active loading % was considered at the following: 0.5% w/w to 10% w/w, 20% w/w, 30% w/w. The melting points cover from 40° C. to 200° C. The wax can be beeswax, fruit or plant wax, but not necessarily limited. In these examples, two different types of waxes and one modified siloxane product were studied: Chemical A: Paraffin Wax; Chemical B: Bath Wax; and Chemical C: TEGO 1345 modified siloxane product, which has PDMS polymers with organic groups grafted on to either the chain ends or the main chain in places.

Table 1 provides a data summary of test results from spunbond barrier/repellency improvement for Inventive Examples A1, A2, A3, B1, B2, C1-TEGO 1345 with Non-FC chemicals compared to 50 gsm natural Control polypropylene spunbond nonwoven without treatment. Inventive Examples A1, A2, A3 were produced with polypropylene forming a single layer of spunbond containing 5%, 10%, 15% weight/weight of active ingredient chemical A, respectively. For Inventive Examples B1 and B2, these were produced with polypropylene forming a single layer of spunbond containing 10% and 14% w/w of active ingredient chemical B, respectively. Inventive Example C1-TEGO 1345 was produced with polypropylene forming a single spunbond layer containing 2.1 w/w of active ingredient chemical C.

TABLE 1

| | | | | | Increase | | Increase % | | Increase of | | Increase of |
|---|---|---|---|---|---|---|---|---|---|---|---|
| | Active | | | Hydro- | of HSH | | of Max IPA | | Ethanol | | LSTST |
| | Ingredients | | | head | % Vs | Max IPA | Repellency | Max | repellency | | % Vs |
| | in SB | BW | AP | Avg | control | Repellency | Vs control | Ethanol | % Vs | LSTST | Control |
| Example | layer (%) | gsm | CFM | mbar | % | % | % | Repellency | Control | sec | % |
| 50 gsm SB | 0 | 44 | 452 | 13.5 | | 20 | | 40 | | 4 | |
| Control | | | | | | | | | | | |
| A1 | 5 | 44 | 422 | 12.5 | — | 30 | 50 | 70 | 75 | 5 | 25 |
| A2 | 10 | 49 | 365 | 13.3 | — | 30 | 50 | 60 | 50 | 7 | 63 |
| A3 | 15 | 58 | 304 | 15 | 12.0 | 30 | 50 | 60 | 50 | 9 | 125 |
| B1 | 10 | 48 | 398 | 14 | 4.7 | 40 | 100 | 60 | 50 | 28 | 600 |
| B2 | 14 | 47 | 410 | 15 | 11.4 | 30 | 50 | 60 | 50 | 14 | 250 |
| C1 - TEGO | 2.1 | 45 | 409 | 11 | — | 30 | 50 | 60 | 50 | 8 | 88 |
| 1345 | | | | | | | | | | | |

The 50 gsm SB control nonwoven was produced using resin-3155 Exxon 35 MFR PP on Berry Nova SB pilot line @ below setup and process conditions.

### Berry Nova Spunbond Pilot Line Equipment Setup:

Nova SB Pack Setup-Single Mono Spinneret Die 183 holes @ 17.2″ HPI.
Nova SB Die Filtration Screen-325 Mesh.
Machine Width Footprint-Laydown formation belt @22.8″
Nova SB Jet setup-Spray swath width @ 8-14″ range.
Nova Mono Extruder Spin pump capacity-Single pump @ 20 cc.

### Process Conditions:

Line speed-23 fpm for target basis wt. of 50 gsm
Nova Mono Extruder temps −230° C.
Nova Feed Pipe temps −230° C.
Nova Beam temps −230° C.
Nova Spin Pump temps −230° C.
Nova Wearplate temps −230° C.
Nova Blocks 1 & 2 temps−230° C.
Nova Mono Extruder pressure-500 psi
Nova Mono Extruder Screw speed range-29.2-42.0-rpm
Jet Air Pressure-20 psi
Nova Auxillary Equipment Setup-Laydown Fan 100% output, Quench Fan-20% output
Nova Charge Bar setup-0.5 Ma-9 KV
Nova Mono Extruder Spin pump setup-14.2 rpm
Total Target Thru-puts-0.625 ghm. Total Thru-put was 6.89 KG/HR
Nova Draw Roll Speed-3,000 rpm DPF-3.0 Denier
Nova Laydown Calendar Surface temps−135° C.
Nova Calendar Nip pressure-800 psi

### C. Meltblown Barrier/Repellency

Table 2 summarizes the test results from meltblown (MB) barrier/repellency improvements for Inventive Examples A4, A5, A6, A7 with Blue color and B3, B4 with blended Non-FC chemicals compared to 34 gsm natural Control MB nonwoven without treatment.

Inventive Examples A4, A5, A6, A7 were produced where polypropylene forming a single layer of MB and contained 5%, 10%, 15%, 10%+3% blue color w/w of active ingredient chemical A, respectively. For Inventive Examples B3, B4, they were produced with polypropylene forming a single layer of MB containing 10%, 15% w/w of active ingredient chemical B, respectively.

TABLE 2

| | | | | | | | Increase % |
|---|---|---|---|---|---|---|---|
| | | | | | Increase of | | of Max IPA |
| | Active | | | Hydro- | HSH | | Repellency |
| | Ingredients | | | head | % Vs | Max IPA | Vs |
| | in MB | BW | AP | Avg | control | Repellency | control |
| Example | layer | gsm | CFM | mbar | % | % | % |
| 34 gsm MB | 0 | 37 | 115 | 41.4 | | 30 | |
| Control | | | | | | | |
| A4 | 5 | 35 | 84 | 54.1 | 30.9 | 40 | 33.3 |
| A5 | 10 | 36 | 62 | 68.1 | 64.7 | 40 | 33.3 |
| A6 | 15 | 41 | 57 | 63.9 | 54.5 | 50 | 66.7 |
| A7 with Blue | 10 + 3% | 36 | 93 | 58.2 | 40.7 | 40 | 33.3 |
| | Blue | | | | | | |
| B3 | 10 | 38 | 54 | 72.2 | 74.6 | 40 | 33.3 |
| B4 | 15 | 37 | 40 | 79.9 | 93.1 | | |
| 44 gsm | | 45 | 32.63 | 77.2 | | 80 | |
| SMMMMS | | | | | | | |
| C6FC AR/AS | | | | | | | |
| treated | | | | | | | |
| (SZT2A215903502) | | | | | | | |

| | | Increase of | | Increase of | Penetration |
|---|---|---|---|---|---|
| | | Ethanol | | LSTST | Test with |
| | Max | repellency | | % Vs | Synthetic |
| | Ethanol | % Vs | LSTST | Control | Blood |
| Example | Repellency | Control | sec | % | g |
| 34 gsm MB | 60 | | 19 | | 0.7 |
| Control | | | | | |
| A4 | 70 | 17 | 73 | 287 | 0.72 |
| A5 | 80 | 33 | 116 | 518 | 0.68 |
| A6 | 80 | 33 | 185 | 883 | 0.68 |
| A7 with Blue | 80 | 33 | 91 | 384 | 0.65 |
| B3 | 70 | 17 | 580 | 2981 | 0.66 |
| B4 | 70 | 17 | 928 | 4825 | 0.69 |
| 44 gsm | 100 | | 942 | | 0.68 |
| SMMMMS | | | | | |
| C6FC AR/AS | | | | | |
| treated | | | | | |
| (SZT2A215903502) | | | | | |

(*AR for MB was done with 2 layers to see if penetrated through 1 layer)
(** Above single MB layer was tested by added a layer of plastic mesh on top of single MB layer.)

The 34 gsm Control MB nonwoven was produced using MB resin-Borealis HL708FB MB Grade 800 MFR PP on Berry Horizon Meltblown line at below setup and process conditions.

### Berry Horizon Meltblown Line Equipment Setup:

Meltblown Die Pack-2,555 Hole Die tip @ 35 HPI″.
Meltblown Die Filtration Screen-325 Mesh.
Machine Width Footprint-Laydown formation belt @ 60″ width.
Meltblown Vertical DCD Cart @45 degree angle to produce 52-54″ Spray width.
Meltblown Spin pump capacity-Dual Spin pumps@30 cc each.

### MB Process Conditions:

Linespeed-27 ypm
Target basis wt. 34-35 gsm
MB Extruder temps −250° C.
MB Die Cavity temps −260° C.
MB Air Heater temps −262° C.
MB Process Air Valves-2.2 psi
Extruder pressure-500 psi
Extruder screw speed range-39.2-45.0 rpms
MB Vertical DCD cart-DCD @13.37″
MB Spin Beam-Dual Spin pumps set @ 25 rpm
Total Target Thru-puts-62.6 KG/HR
MB Laydown Suction Fan-100% output

### D. Meltblown Barrier/Repellency and Anti-Static Treatment

Table 3 summarizes test results from MB barrier/repellency improvement with the addition of an anti-static treatment. Inventive Example A8 was produced with polypropylene forming single layer of MB and contained 10% w/w of active ingredient chemical A then kiss coating with added 0.2% anionic anti-static treatment compared to 34 gsm Control single MB layer nonwoven kiss coating with added 0.1% anionic anti-static treatment.

TABLE 3

| | | | | | Increase of | | Increase % | |
|---|---|---|---|---|---|---|---|---|
| | Active | | | Hydro- | HSH | | of Max IPA | |
| | Ingredients | | | head | % Vs | Max IPA | Repellency | Max |
| | in each | BW | AP | Avg | control | Repellency | Vs control | Ethanol |
| Example | layer | gsm | CFM | mbar | % | % | % | Repellency |
| 34 gsm MB Control | 0 + ANT | 36.7 | 105 | 36 | | 20 | | 40 |
| with anti-static | 0.1% | | | | | | | |
| treated | | | | | | | | |
| A8 | 10 + ANT | 36.5 | 62 | 75.4 | 109 | 30 | 50 | 60 |
| with anti-static | 0.2% | | | | | | | |
| treated | | | | | | | | |
| (LW01152024-D) | | | | | | | | |

| | Increase of | | Increase of | Penetration | | |
|---|---|---|---|---|---|---|
| | Ethanol | | LSTST | Test with | | |
| | repellency | | % Vs | Synthetic | 50% RH/ | 50% RH/ |
| | % Vs | LSTST | Control | Blood | 50% cut-off | 10% cut-off |
| Example | Control | secs | % | g | sec | sec |
| 34 gsm MB Control | | 16 | | 0.9 | 0.01 | 0.03 |
| with anti-static | | | | | | |
| treated | | | | | | |
| A8 | 50 | 137 | 756 | 0.7 | 0.03 | 0.3 |
| with anti-static | | | | | | |
| treated | | | | | | |
| (LW01152024-D) | | | | | | |

(*AR for MB was done with 2 layers to see if penetrated through 1 layer)
(** Above single MB layer was tested by added a layer of plastic mesh on top of single MB layer.)

The static decay was tested using the standard test method IST 40.2 performed at 50% RH using 50% and 10% remaining charge as cut-off level. The properties of this resulting fabric (Examples A8 and 34 gsm Control MB nonwoven) are summarized in Table 3 above. A static decay at 50% RH with 50% cut-off need to be less than 1 second with Target 0.5 second.

### E. Liquid Absorptive Capacity and Run Off Percentage

Table 4 summarizes a group of nonwoven fabrics that were subjected to testing for their (i) liquid absorptive capacity for both a 0.9% NaCl solution and a Triton-X-100 solution, and (ii) liquid run off percentage for both the 0.9% NaCl solution and a Triton-X-100 solution. Each of the liquid absorptive capacity tests were conducting according to NWSP 10.1.R0(20) (Clause b) following Section 8.2: The liquid absorptive capacity and where and mass of solution that is absorbed by the mass of the tested nonwoven fabric is expressed as a percentage of the mass of the test nonwoven fabric. Each of the run off tests were conducted according to Standard Procedure: NWSP 080.9.R0 (15), Option A-Basic Method.

TABLE 4

| | Liquid | Liquid | | |
|---|---|---|---|---|
| | Absorptive | Absorptive | | |
| Data Summary | Capacity | Capacity | Run Off | Run Off |
| Sample ID | 0.9% NaCl | Triton-X 100 | 0.9% NaCl | Triton-X 100 |
| | | (32 dyne surface | | (32 dyne surface |
| | | tension) | | tension) |
| | | Solution | | |
| 34 gsm MB Control in Table 2 | 35.24 | 504.90 | 98.87 | 90.50 |
| Example A5 in Table 2 (10% w/w of | 19.04 | 293.82 | 98.36 | 94.16 |
| active ingredient chemical A in [symbol] | | | | |
| Example B3 in Table 2 (10% w/w of | 12.25 | 164.46 | 97.92 | 95.24 |
| active ingredient chemical B in [symbol] | | | | |
| SZB1493095AA201 (Control base) | 71.06 | 445.58 | 98.43 | 84.85 |
| (without treatment) | | | | |
| Example SZB1493094AA201 | 19.46 | 209.03 | 98.80 | 92.28 |
| (S/M/M/M/S = A/B/C/D/E = | | | | |
| 0/10/10/10/10% w/w of active | | | | |
| ingredient chemical A in each layer) | | | | |
| Example SZB1493094AA202 | 20.98 | 212.78 | 98.39 | 91.56 |
| (S/M/M/M/S = A/B/C/D/E = | | | | |
| 0/10/10/10/10% w/w of active | | | | |
| ingredient chemical A in each layer) | | | | |
| 44 gsm SMMMMS C6FC AR/AS treated | 50.19 | 69.99 | 98.75 | 97.21 |
| made on S3 China (SZT2A215903502) | | | | |

[symbol]

indicates data missing or illegible when filed

As can be seen from the data from Table 4, the nonwoven fabrics treated with a NFLST additive exhibited a noticeably lower absorptive capacity (e.g., from about ½ to about ⅓ of a non-treated comparative/control sample), which indicates that these nonwoven fabrics would provide a faster drying rate and/or time (e.g., drying capability). In this regard, such nonwoven fabrics may desirably be used as a component of an article, such as an ostomy bag and/or wound care articles.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A barrier fabric, comprising: at least a first nonwoven layer, the first nonwoven comprising a plurality of first fibers defined by a first polymeric composition, wherein the first polymeric composition includes (i) a first polymer component, and (ii) a first additive component comprising at least one non-fluorinated low-surface tension (NFLST) additives dispersed throughout the first polymeric composition.

2. The fabric of claim 1, wherein the NFLST additives include one or more waxes, such as a paraffin wax, a glycerol tri-stearate, a beeswax, a cuticular wax, or any combination thereof.

3. The fabric of claim 1, wherein the NFLST additives include an organo-modified siloxane.

4. The fabric of claim 3, wherein the organo-modified siloxane is selected from Formula (I): 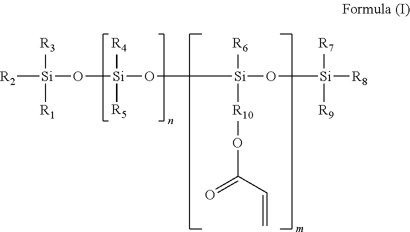
wherein,
R1-R9 are independently selected from —H, a C1-C10 radical, —OH, alkoxy radical, and an acrylate functional group,
R10 is selected from a C1-C10 hydrocarbon;
n is selected from 1 to about 100, and
m is selected from 1 to about 100.

5. The fabric of claim 3, wherein the organo-modified siloxane is selected from Formula (II): 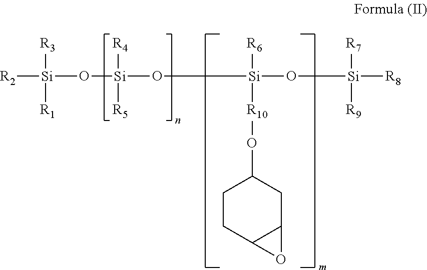
wherein
R₁-R₉ are independently selected from —H, a C1-C10 radical, —OH, alkoxy radical, and an acrylate functional group,
R10 is selected from a C1-C10 hydrocarbon;
n is selected from 1 to about 100, and
m is selected from 1 to about 100.

6. The fabric of claim 1, wherein the NFLST additives include an acrylic-functional polymer including an alkyl silane methacrylate group.

7. The fabric of claim 6, wherein the acrylic-functional polymer is cationic.

8. The fabric of claim 1, wherein the first plurality of fibers comprise spunbond fibers, meltblown fibers, or staple fibers.

9. The fabric of claim 1, wherein the first nonwoven layer is a first spunbond layer defining a first outermost layer, and wherein the first plurality of fibers comprise a first plurality of continuous spunbond fibers, the fabric further comprising (i) a second nonwoven layer comprising a second spunbond layer defining a second outermost layer including a second plurality of continuous spunbond fibers, and (ii) a plurality of inner fine fiber-containing nonwoven layers including a first fine fiber-containing layer including a first plurality of fine fibers, wherein the plurality of inner fine fiber-containing nonwoven layers are located directly or indirectly between the first spunbond layer and the second spunbond layer.

10. The fabric of claim 9, wherein the second spunbond layer includes a second polymeric composition including (i) a second polymer component, and (ii) a second additive component comprising at least one NFLST additive dispersed throughout the second polymeric composition.

11. The fabric of claim 9, wherein the first fine fiber-containing layer includes a third polymeric composition including (i) a third polymer component, and (ii) a third additive component comprising at least one NFLST additive dispersed throughout the third polymeric composition.

12. The fabric of claim 9, wherein the barrier fabric comprises one of the following structures:
(Structure 1) S1ₐ-M_{b}-S2_{c};
(Structure 2) S1ₐ-N_{d}—S2_{c};
(Structure 3) S1ₐ-M_{b}-N_{d}—S2_{c};
(Structure 4) S1ₐ-N_{d}-M_{b}-N_{d}—S2_{c}
(Structure 5) S1ₐ-M_{b}-N_{d}-M_{b}-S2_{c}; or any combinations thereof;
wherein
‘M’ comprises a meltblown layer or a melt-fibrillated layer;
‘N’ comprises a sub-micron fiber-containing layer, such as electrospun fibers;
‘S1’ comprises a first spunbond layer;
‘S2’ comprises a second spunbond layer;
‘a’ represents the number of layers and is independently selected from 1, 2, 3, 4, and 5;
‘b’ represents the number of layers is independently selected from 1, 2, 3, 4, 5, 6, 7, and 8;
‘c’ represents the number of layers is independently selected from 1, 2, 3, 4, and 5; and
‘d’ represents the number of layers is independently selected from 1, 2, 3, 4, 5, 6, 7, and 8;
‘e’ represents the number of layers is independently selected from 1, 2, 3, 4, and 5.

13. The nonwoven fabric of claim 1, wherein the NFLST additives comprises a first ratio between a total wax content and a total organo-modified siloxane content on a dry basis (% wt.:% wt.) from about 1:10 to about 10:1.

14. The nonwoven fabric of claim 1, wherein the NFLST additives comprises a second ratio between a total wax content and a total acrylic-functional polymer including an alkyl silane methacrylate group content on a dry basis (% wt.:% wt.) from about 1:10 to about 10:1.

15. The fabric of claim 1, wherein the barrier fabric has one or more of the following: (i) a hydrohead from about 40 mbar; (ii) an air permeability of 5 CFM or greater according to IST70.1; (iii) a liquid strike through time (LSTST) from about 5 to about 1500 seconds; and (iv) an IPA repellency % from about 20 to about 60%.

16. An article, comprising:
(i) a backsheet comprising a barrier fabric according to claim 1;
(ii) a liquid permeable topsheet; and
(iii) an absorbent core located between the backsheet and the liquid permeable topsheet.

17. A method of forming a barrier fabric, comprising:
(i) forming a first polymer melt comprising a first polymeric composition including (a) a first polymer component, and (b) a first additive component, wherein the first additive component includes at least one non-fluorinated low-surface tension (NFLST) additives dispersed throughout the first polymeric composition;
(ii) meltspinning the first polymer melt to provide a first plurality of fibers; and
(iii) consolidating the first plurality of fibers to provide the barrier fabric9.

18. The method of claim 17, further comprising (i) forming a second polymer melt comprising a second polymeric composition including (a) a second polymer component, and (b) a second additive component, wherein the second additive component includes at least one non-fluorinated low-surface tension (NFLST) additives dispersed throughout the second polymeric composition, and (ii) meltspinning the second polymer melt via a spunbond process to form a second plurality of continuous spunbond fibers.

19. The method of claim 17, further comprising (i) forming a third polymer melt comprising a third polymeric composition including (a) a third polymer component, and (b) a third additive component, wherein the third additive component includes at least one non-fluorinated low-surface tension (NFLST) additives dispersed throughout the third polymeric composition, and (ii) meltspinning the second polymer melt via a meltblown process to form a plurality of meltblown fibers.

20. The method of claim 19, further comprising a step of depositing the plurality of meltblown fibers onto the first plurality of continuous spunbond fibers, and depositing the second plurality of continuous spunbond fibers onto the plurality of meltblown fibers.
